SEQUENCE LISTING (28 sequences)

SEQ ID NO: 1 - DNA, 1248 bp, synthetic construct
  source 1..1248: mol_type = other DNA
atgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactctga

SEQ ID NO: 2 - protein, 415 aa, synthetic construct
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 3 - DNA, 1248 bp, synthetic construct
  source 1..1248: mol_type = other DNA
atgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcagcatcccctacagtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactctga

SEQ ID NO: 4 - protein, 415 aa, synthetic construct
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSSIPYSQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 5 - DNA, 1344 bp, synthetic construct
  source 1..1344: mol_type = other DNA
atgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgtgagtgcctcatgggtggggtcagcagggagagccctgtgtttagacaaggcctgggcagagaccctctctccctgactctcctgctctccccaggagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactctga

SEQ ID NO: 6 - DNA, 1443 bp, synthetic construct
  source 1..1443: mol_type = other DNA
atgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactga

SEQ ID NO: 7 - protein, 480 aa, synthetic construct
MIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 8 - DNA, 54 bp, synthetic construct
  source 1..54: mol_type = other DNA
atgaagtgggtaacctttatttcccttctttttctctttagctcggcttattcc

SEQ ID NO: 9 - DNA, 1413 bp, synthetic construct
  source 1..1413: mol_type = other DNA
atgaagtgggtaacctttatttcccttctttttctctttagctcggcttattccgcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactga

SEQ ID NO: 10 - protein, 470 aa, synthetic construct
MKWVTFISLLFLFSSAYSAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 11 - DNA, 545 bp, synthetic construct
  source 1..545: mol_type = other DNA
cccgggaggcagaggttgcagtgagccgagatcgcgccactgcactccaacctgggtgacagactctgtctccaaaacaaaacaaacaaacaaaaagattttattaaagatattttgttaactcagtaaatctgtgactcattctctccacgacgacaggcggcctttttttttccgcgcccggctctttaaaatcggccccgcccctgtgacgtgtcctggtgccgcggccaatcaaaaagcccaataaggatggtgccagtcccttcttcaccccagccagtccgcttcccgttcgggtcacgcgctccgggcctccaagagggcctccaagagggccaatcggaagggcaagcttcgagatgctgcgtgatcacgtgggaggagatgagctacttgaagaccaattagagtccgggaagcgcggcggggcctccagaccggggcgggcttaagggtgacatctgcgctttaaagggtccgggtcagctgactcccgactctgtggagtctagctgccagggtcgcggcagctgcggggagag

SEQ ID NO: 12 - DNA, 321 bp, synthetic construct
  source 1..321: mol_type = other DNA
ccgcggccaatcaaaaagcccaataaggatggtgccagtcccttcttcaccccagccagtccgcttcccgttcgggtcacgcgctccgggcctccaagagggcctccaagagggccaatcggaagggcaagcttcgagatgctgcgtgatcacgtgggaggagatgagctacttgaagaccaattagagtccgggaagcgcggcggggcctccagaccggggcgggcttaagggtgacatctgcgctttaaagggtccgggtcagctgactcccgactctgtggagtctagctgccagggtcgcggcagctgcggggagag

SEQ ID NO: 13 - DNA, 663 bp, synthetic construct
  source 1..663: mol_type = other DNA
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 14 - DNA, 493 bp, synthetic construct
  source 1..493: mol_type = other DNA
cgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggat

SEQ ID NO: 15 - DNA, 96 bp, synthetic construct
  source 1..96: mol_type = other DNA
gtgagtgcctcatgggtggggtcagcagggagagccctgtgtttagacaaggcctgggcagagaccctctctccctgactctcctgctctccccag

SEQ ID NO: 16 - DNA, 579 bp, synthetic construct
  source 1..579: mol_type = other DNA
ccgcccctctccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 17 - DNA, 174 bp, synthetic construct
  source 1..174: mol_type = other DNA
tgacaatggcgactagcctcccaagccagccacccagggcgagtcatcgacccaaaaggtcagaccgtctcacacaaacaatcccaagtaaaggctctgacgtctccccctttttttaggaactgaaaccacggccctgacgtccctcccccctaggaacaggaacagctctcc

SEQ ID NO: 18 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 19 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 20 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 3791 bp, synthetic construct
  source 1..3791: mol_type = other DNA
cccgggaggcagaggttgcagtgagccgagatcgcgccactgcactccaacctgggtgacagactctgtctccaaaacaaaacaaacaaacaaaaagattttattaaagatattttgttaactcagtaaatctgtgactcattctctccacgacgacaggcggcctttttttttccgcgcccggctctttaaaatcggccccgcccctgtgacgtgtcctggtgccgcggccaatcaaaaagcccaataaggatggtgccagtcccttcttcaccccagccagtccgcttcccgttcgggtcacgcgctccgggcctccaagagggcctccaagagggccaatcggaagggcaagcttcgagatgctgcgtgatcacgtgggaggagatgagctacttgaagaccaattagagtccgggaagcgcggcggggcctccagaccggggcgggcttaagggtgacatctgcgctttaaagggtccgggtcagctgactcccgactctgtggagtctagctgccagggtcgcggcagctgcggggagagatgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactctgactcgagccgcccctctccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaagtgggtaacctttatttcccttctttttctctttagctcggcttattccgcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactga

SEQ ID NO: 23 - protein, 18 aa, synthetic construct
EGRGSLLTCGDVEENPGP

SEQ ID NO: 24 - protein, 19 aa, synthetic construct
ATNFSLLKQAGDVEENPGP

SEQ ID NO: 25 - protein, 20 aa, synthetic construct
QCTNYALLKLAGDVESNPGP

SEQ ID NO: 26 - protein, 22 aa, synthetic construct
VKQTLNFDLLKLAGDVESNPGP

SEQ ID NO: 27 - DNA, 20 bp, synthetic construct
  source 1..20: mol_type = other DNA
cctggcctcggattccgccg

SEQ ID NO: 28 - DNA, 20 bp, synthetic construct
  source 1..20: mol_type = other DNA
atatccacagagagattccg

(19) United States
(12) Patent Application Publication
(10) Pub. No.: US 2026/0092290 A1
(43) Pub. Date: Apr. 2, 2026

(54) RECOMBINANT VIRAL VECTOR, RECOMBINANT ADENO-ASSOCIATED VIRUS COMPRISING THE SAME, AND USES THEREOF IN TREATING SIALIDOSIS

(71) Applicant: National Taiwan University, Taipei City (TW)

(72) Inventor: Wuh-Liang HWU, Taipei City (TW)

(21) Appl. No.: 19/112,675

(22) PCT Filed: Oct. 18, 2023
(86) PCT No.: PCT/CN2023/125117

§ 371 Date: Mar. 18, 2025

Related U.S. Application Data
(60) Provisional application No. 63/434,096, filed on Dec. 21, 2022.

(51) Int. Cl. C12N 15/86; A61K 38/00; A61K 48/00; A61P 3/00; C12N 9/24; C12N 9/48

(52) U.S. Cl. CPC C12N 15/86; A61K 48/0066; A61P 3/00; C12N 9/2402; C12N 9/485; A61K 38/00; C12N 2750/14143; C12N 2800/22; C12N 2830/50; C12N 2840/203; C12Y 302/01018; C12Y 304/16006

(57) ABSTRACT

Disclosed herein is a recombinant viral vector comprising an AAV 5′ inverted terminal repeat (ITR) sequence, an AAV 3′ ITR sequence, and an expression cassette flanked by the AAV 5′ ITR and 3′ ITR sequences. According to the embodiments of the present disclosure, the expression cassette comprises a first and a second coding sequences respectively encoding a human neuraminidase 1 (NEU1) and a human protective protein/cathepsin A (PPCA). Also disclosed herein are a recombinant adeno-associated virus (AAV) comprising the recombinant viral vector, a pharmaceutical composition comprising the recombinant AAV, and uses thereof in treating sialidosis.

### CROSS-REFERENCES TO RELATED APPLICATIONS

This application is a U.S. National Stage Filing under 35 U.S.C. from International Patent Application Serial No. PCT/CN2023/125117, filed Oct. 18, 2023, and published on Jun. 27, 2024, and claims the benefit of U.S. Provisional Application No. 63/434,096 filed Dec. 21, 2022, the disclosure of which are incorporated by reference herein in its entireties.

### SEQUENCE LISTING XML

The present application is being filed along with a Sequence Listing XML in electronic format. The Sequence Listing XML is provided as a XML file entitled P4311-PCT_seq listing, created Oct. 11, 2023, which is 42 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to the treatment of sialidosis by use of novel recombinant adeno-associated virus (rAAV).

### 2. Description of Related Art

Sialidosis is a rare lysosomal storage disease caused by the mutation of neuraminidase 1 (NEU) gene that leads to a deficiency of neuraminidase 1 (also known as “sialidase”) in the affected subjects. Deficiency of neuraminidase 1 results in the abnormal accumulation of sialylated glycoconjugates in the body. Sialidosis is divided into two types (i.e., type I and type II sialidosis). Type I sialidosis is the milder, late-onset form of the disease caused by the attenuation of neuraminidase 1 activity, which is characterized by progressive myoclonus, cerebellar ataxia, visual impairment (visual disturbances) and gait abnormality. Over time, some patients develop seizures and mild cognitive impairment. Patients with type I sialidosis usually develop symptoms in their teens or twenties. Type II sialidosis is the severe, early-onset form of the disease caused by a complete loss of neuraminidase 1 activity, which often begins during infancy or childhood, and is characterized by bone dysplasia, coarse faces, organomegaly, severe ataxia and mental retardation. Sialidosis is inherited as an autosomal recessive trait. The macular cherry red spot is found in both forms.

Currently, there is no treatment for sialidosis. Short-term, high-dose enzyme replacement therapy (ERT) in sialidosis mice was examined in 2005; however, after 2 weeks of ERT, the mice developed a severe immune response towards the exogenous NEU1 enzyme. In view of the foregoing, there is a continuing interest in developing a novel agent and method for treating sialidosis.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the disclosure is directed to a recombinant viral vector, which comprises an AAV 5′ inverted terminal repeat (ITR) sequence, an AAV 3′ ITR sequence, and an expression cassette flanked by the AAV 5′ ITR and 3′ ITR sequences. According to the embodiments of the present disclosure, the expression cassette comprises,
(a) a promoter;
(b) a first coding sequence operably linked to the promoter encoding a human neuraminidase 1 (NEU1); and
(c) a second coding sequence operably linked to the promoter encoding a human protective protein/cathepsin A (PPCA).

According to some embodiments, the AAV 5′ ITR and 3′ ITR sequences are derived from AAV serotype 2 (AAV2). In one exemplary embodiment, the AAV 5′ ITR and 3′ ITR sequences respectively comprise the nucleotide sequences of SEQ ID NOs: 19 and 20.

Depending on desired purpose, the promoter may be a human NEU1 promoter (e.g., human P2 NEU1 promoter or human P3 NEU1 promoter), or a cytomegalovirus (CMV) promoter. In one preferred embodiment, the promoter is a human NEU1 P3 promoter, and comprises the nucleotide sequence of SEQ ID NO: 11.

According to certain embodiments, the human NEU1 and human PPCA respectively comprise the amino acid sequences of SEQ ID NOs: 2 and 10. In one preferred embodiment, the first and second coding sequences respectively comprise the nucleotide sequences of SEQ ID NOs: 1 and 9.

Preferably, the expression cassette further comprises an internal ribosomal entry site (IRES) disposed between the first and second coding sequences. Alternatively, the expression cassette further comprises a third coding sequence disposed between the first and second coding sequences, wherein the third coding sequence encodes a 2A peptide.

According to some embodiments, the expression cassette further comprises a polyadenylation (polyA) sequence disposed at the 3′ end of the second coding sequence. In one preferred embodiment, the polyA sequence is derived from human growth hormone (hGH).

According to one specific embodiment, the expression cassette comprises the nucleotide sequence of SEQ ID NO: 22.

Also disclosed therein is a recombinant AAV, which comprises in its structure, an AAV capsid having the recombinant viral vector of the present disclosure packaged therein. Preferably, the AAV capsid has a broad tropism for the brain and visceral organs. According to some exemplary embodiment, the AAV capsid is derived from AAV serotype 9 (AAV9). In one working example, the AAV capsid comprises the amino acid sequence of SEQ ID NO: 21.

Another aspect of the present disclosure pertains to the use of the recombinant AAV for the preparation of a pharmaceutical composition, and uses thereof in the treatment of sialidosis.

According to some embodiments of the present disclosure, the pharmaceutical composition comprises the present recombinant AAV and a pharmaceutically acceptable carrier.

The recombinant AAV or pharmaceutical composition is useful in treating sialidosis. Accordingly, also disclosed herein is a method of treating sialidosis in a subject. The method comprises administering to the subject an effective amount of the recombinant AAV or pharmaceutical composition, so as to alleviate or ameliorate the symptoms of the sialidosis.

Depending on intended purpose, the sialidosis may be type I sialidosis or type II sialidosis.

According to some embodiments, about 1×10¹³ to 1×10¹⁵ genome copy (GC)/kg brain weight of the recombinant AAV is administered to the subject. According to some embodiments, about 1×10¹³ to 1×10¹⁵ genome copy (GC)/kg body weight of the recombinant AAV is administered to the subject.

The recombinant AAV or pharmaceutical composition may be administered to the subject via a suitable route, for example, intra-cerebrospinal fluid (e.g., intracerebral ventricular, intracisternal or intrathecal), intravenous, intraperitoneal, intraarterial, or intramuscular injection. According to one embodiment, the recombinant AAV or pharmaceutical composition is administered to the subject via intra-cerebrospinal fluid injection. According to another embodiment, the recombinant AAV or pharmaceutical composition is administered to the subject via intravenous injection.

Among all embodiments of the present disclosure, the subject is a mammal; preferably, a human.

Many of the attendant features and advantages of the present disclosure will becomes better understood with reference to the following detailed description.

### DETAILED DESCRIPTION OF THE INVENTION

The detailed description provided below is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. DEFINITION

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “viral vector” refers to a synthetic or artificial viral nucleic acid, in which an expression cassette containing a gene of interest (e.g., the first and second coding sequences of the present disclosure) is packaged in a viral capsid or an envelope, where any viral genomic sequences also packaged within the viral capsid or envelope are replication-deficient; i.e., they cannot generate progeny virions but retain the ability to infect target cells and express the gene of interest in the infected target cells. In one embodiment, the genome of the viral vector does not include genes encoding the enzymes required to replicate (the genome can be engineered to be “gutless” that contains only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Alternatively, the term “viral vector” may refer to a virus or viral particle that mediates the transfer of a nucleic acid molecule into a cell. In addition to the nucleic acid molecule, the virus or viral particle typically includes various viral components and optionally host cell components.

As used herein, the term “recombinant viral vector” refers to a viral vector comprising one or more heterologous gene products or sequences. In general, the heterologous gene products or sequences are introduced by replacing one or more regions of the viral genome.

The term “AAV vector” as used herein refers to a synthetic or artificial viral nucleic acid having an AAV 5′ inverted terminal repeat (ITR) sequence, an AAV 3′ ITR, and a gene of interest (e.g., the first and second coding sequences of the present disclosure) flanked by the AA5 5′ and 3′ ITRs. In general, the gene of interest is operably linked to transcription regulatory elements, e.g., promoter, enhancer, polyadenylation (polyA) sequence, and optionally, one or more introns inserted between exons of the gene of interest.

The term “inverted terminal repeat” (ITR) refers to the art-recognized regions found at the 5′ and 3′ termini of an AAV genome that are required in cis for the vector genome replication and its packaging into viral particles. They are also needed for viral genome integration into host genome, for the rescue from the host genome, and for the encapsulation of viral nucleic acid into mature virions.

As used herein, the term “operably linked” refers to both expression control sequences that are contiguous with the gene of interest (e.g., the first and second coding sequences of the present disclosure) and expression control sequences (e.g., the promoter of the present disclosure) that act in trans or at a distance to control the gene of interest.

The term “derived from” as used herein refers to an origin or source of a molecule (e.g., polynucleotide or polypeptide). The term “derived from” with respect to a polynucleotide or polypeptide of the invention being derived from a particular origin or source, means that the polynucleotide or polypeptide has the same sequence as a polynucleotide or polypeptide found naturally in that origin or source. Alternatively, the polynucleotide or polypeptide of the invention may be a fragment of the naturally occurring polynucleotide or polypeptide. The polynucleotide or polypeptide, derived from a particular origin or source, may be produced via direct cloning, PCR amplification, or artificial synthesis from, or based on, a naturally occurring sequence.

As discussed herein, minor variations in the amino acid sequences of polypeptides or in the nucleic acids of polynucleotides are contemplated as being encompassed by the presently disclosed and claimed inventive concept(s), providing that the variations in the amino acid sequence/nucleic acid maintain at least 80% sequence identity, such as at least 80%, 81%, 82%, 83%, 84%, 85%, 86%, 87%, 88%, 89%, 90%, 91%, 92%, 93%, 94%, 95%, 96%, 97%, 98% and 99% sequence identity. Polypeptides or polynucleotides of the present disclosure may be modified specifically to alter a feature of the polypeptide or polynucleotide unrelated to its physiological activity. For example, certain amino acids can be changed and/or deleted without affecting the physiological activity of the polypeptide in this study. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site.

“Percentage (%) sequence identity” is defined as the percentage of amino acid residues/nucleic acids in a candidate sequence that are identical with the amino acid residues/nucleic acids in the specific polypeptide/polynucleotide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences/nucleic acids was carried out by computer program Blastp (protein-protein BLAST)/Blastn (nucleotide-nucleotide BLAST) provided online by Nation Center for Biotechnology Information (NCBI). The percentage amino acid sequence/nucleic acid identity of a given amino acid sequence/nucleic acid A to a given amino acid sequence/nucleic acid B (which can alternatively be phrased as a given amino acid sequence/nucleic acid A that has a certain % amino acid sequence/nucleic acid identity to a given amino acid sequence/nucleic acid B) is calculated by the formula as follows:

$$\frac{X}{Y} \times 100$$

where X is the number of amino acid residues/nucleic acids scored as identical matches by the sequence alignment program BLAST in that program's alignment of A and B, and where Y is the total number of amino acid residues/nucleic acids in A or B, whichever is shorter.

As used herein, the term “treat,” “treating” and “treatment” are interchangeable, and encompasses partially or completely preventing, ameliorating, mitigating and/or managing a symptom, a secondary disorder or a condition associated with sialidosis. The term “treating” as used herein refers to application or administration of the recombinant AAV or pharmaceutical composition of the present disclosure to a subject, who has a symptom, a secondary disorder or a condition associated with sialidosis, with the purpose to partially or completely alleviate, ameliorate, relieve, delay onset of, inhibit progression of, reduce severity of, and/or reduce incidence of one or more symptoms, secondary disorders or features associated with sialidosis. Symptoms, secondary disorders, and/or conditions associated with sialidosis include, but are not limited to, macular cherry red spot, progressive myoclonus, ataxia, visual impairment (visual disturbances), gait abnormality, cognitive impairment, bone dysplasia, coarse faces, and organomegaly. Treatment may be administered to a subject who exhibits only early signs of such symptoms, disorder, and/or condition for the purpose of decreasing the risk of developing the symptoms, secondary disorders, and/or conditions associated with sialidosis. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. According to some embodiments of the present disclosure, the effective amount is administered to the subject in one dose. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, as grams, milligrams or micrograms; as milligrams per kilogram of body weight (mg/Kg). Alternatively, effective amount may be expressed as genome copy per kilogram of body weight (GC/kg). Persons having ordinary skills could calculate the human equivalent dose (HED) for the medicament (such as the present recombinant AAV or pharmaceutical composition) based on the doses determined from animal models. For example, one may follow the guidance for industry published by U.S. Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” refers to an animal including the human species that is treatable by the recombinant AAV, pharmaceutical composition and/or method of the present invention. The term “subject” is intended to refer to both the male and female gender unless one gender is specifically indicated.

### II. DESCRIPTION OF THE INVENTION

The present disclosure aims at providing a recombinant AAV for treating sialidosis. The recombinant AAV of the present invention is characterized by carrying two therapeutic genes, NEU1 and CTSA, in its viral genome. After administering to sialidosis patients, the recombinant AAV simultaneously expresses human neuraminidase 1 (hNEU1) and human protective protein/cathepsin A (hPPCA) in the patients to effectively alleviate or ameliorate the symptoms of the sialidosis. Accordingly, also disclosed herein are a viral vector for expressing the hNEU1 and hPPCA, and methods of treating sialidosis by using the recombinant AAV.

### (i) Recombinant AAV Vector

The first aspect of present disclosure is directed to a recombinant AAV vector, which comprises, in the order from 5′ end to 3′ end,
(a) an AAV 5′ ITR sequence;
(b) an expression cassette comprising,
   (b-1) a promoter;
   (b-2) a hNEU1-coding sequence operably linked to the promoter; and
   (b-3) a hPPCA-coding sequence operably linked to the promoter; and
(c) an AAV 3′ ITR sequence.

As known in the art, the ITR sequences are the genetic elements responsible for the replication and packaging of the genome during viral production and are the only viral cis elements required to generate rAAV. The minimal sequences required to package the expression cassette into an AAV viral particle are the AAV 5′ ITR and 3′ ITR sequences, which may be of the same AAV origin as the capsid, or of a different AAV origin from the capsid (to produce an AAV pseudotype). Depending on intended purpose, the AAV 5′ ITR and 3′ ITR sequences of the recombinant AAV vector may be derived from AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8) or AAV serotype 9 (AAV9). According to some embodiments of the present disclosure, the present AAV 5′ ITR and 3′ ITR sequences are derived from AAV2, and respectively comprise the nucleotide sequences at least 85% (e.g., 85%, 86%, 87%, 88%, 89%, 90%, 91%, 92%, 93%, 94%, 95%, 96%, 97%, 98%, 99% or 100%) identical to SEQ ID NOs: 19 and 20. Preferably, the present AAV 5′ ITR and 3′ ITR sequences respectively comprise the nucleotide sequences at least 90% identical to SEQ ID NOs: 19 and 20; more preferably, at least 95% identical to SEQ ID NOs: 19 and 20. In one exemplary embodiment, the present AAV 5′ ITR and 3′ ITR sequences respectively comprise the nucleotide sequences 100% identical to SEQ ID NOs: 19 and 20 (i.e., respectively comprising the nucleotide sequences of SEQ ID NOs: 19 and 20).

Depending on desired purpose, the promoter may be a human NEU1 promoter, CMV promoter, elongation factor 1a (EF1a) promoter, simian virus 40 (SV40) promoter, rous sarcoma virus (RSV) promoter, mouse mammary tumor virus (MMTV) promoter, phosphoglycerate kinase (PGK) promoter, chicken beta-active promoter, human H1 promoter, U6 promoter, or any promoters known to constitutively drive the expression of the gene of interest in eukaryotic cells.

According to some exemplary embodiments of the present disclosure, the promoter is a P3 NEU1 promoter, and comprises a nucleotide sequence at least 85% identical to SEQ ID NO: 11; preferably, at least 90% identical to SEQ ID NO: 11; more preferably, at least 95% identical to SEQ ID NO: 11. In one specific example, the promoter comprises a nucleotide sequence 100% identical to SEQ ID NO: 11 (i.e., comprising the nucleotide sequence of SEQ ID NO: 11).

According to certain exemplary embodiments of the present disclosure, the promoter is a P2 NEU1 promoter, and comprises a nucleotide sequence at least 85% identical to SEQ ID NO: 12; preferably, at least 90% identical to SEQ ID NO: 12; more preferably, at least 95% identical to SEQ ID NO: 12. In one specific example, the promoter comprises a nucleotide sequence 100% identical to SEQ ID NO: 12 (i.e., comprising the nucleotide sequence of SEQ ID NO: 12).

According to alternative embodiments of the present disclosure, the promoter is a CMV promoter, and comprises a nucleotide sequence at least 85% identical to SEQ ID NO: 13; preferably, at least 90% identical to SEQ ID NO: 13; more preferably, at least 95% identical to SEQ ID NO: 13. In one specific example, the promoter comprises a nucleotide sequence 100% identical to SEQ ID NO: 13 (i.e., comprising the nucleotide sequence of SEQ ID NO: 13).

According to some embodiments, the hNEU1-coding sequence comprises the nucleotide sequence of SEQ ID NO: 1, and the thus-produced hNEU1 protein comprises the amino acid sequence of SEQ ID NO: 2. As could be appreciated, the present hNEU1-coding sequence may comprise one or more degenerate nucleotides as long as the protein (i.e., the hNEU1 protein) encoded by the degenerate nucleotide sequence maintains the desired activity or function. The term “degenerate nucleotide sequence” (also known as “nucleotide degeneracy”) denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp). Additively or alternatively, the present hNEU1-coding sequence may comprise one or more optimized codon (i.e., codon optimization) to increase hNEU1 protein expression in cells. Accordingly, the nucleotide sequences comprising degenerate nucleotide(s) and/or optimized nucleotide(s) are intended to be included within the scope of the present disclosure, providing that the variations in the nucleotide sequence maintain at least 85% sequence identity to SEQ ID NO: 1, such as at least 85%, 86%, 87%, 88%, 89%, 90%, 91%, 92%, 93%, 94%, 95%, 96%, 97%, 98% or 99% sequence identity to SEQ ID NO: 1.

Alternatively, the hNEU1 protein may be modified to comprise one or more mutations in its amino acid sequence thereby decreasing self-aggregation. For example, according to certain exemplary embodiments, the modified hNEU1 protein comprises two mutations in its amino acid sequence as compared to the hNEU1 protein of SEQ ID NO: 2, in which each amino acid residue “G” at positions 248 and 252 of the hNEU1 protein (SEQ ID NO: 2) is substituted with an amino acid residue “S”, so as to reduce the extent/level of self-aggregation. In the embodiments, the modified hNEU1 protein comprises the amino acid sequence of SEQ ID NO: 4. According to some exemplary embodiments, the modified hNEU1 protein of SEQ ID NO: 4 is encoded by a coding sequence comprising the nucleotide sequence of SEQ ID NO: 3. As described above, the coding sequence encoding the modified hNEU1 may comprise one or more degenerate nucleotides and/or optimized nucleotides as long as the protein (i.e., the modified hNEU1 protein) encoded thereby maintains the desired activity or function. Accordingly, the nucleotide sequences comprising degenerate nucleotide(s) and/or optimized nucleotide(s) are contemplated in the scope of the present disclosure, providing that the variations in the nucleotide sequence maintain at least 85% sequence identity to SEQ ID NO: 3, such as at least 85%, 86%, 87%, 88%, 89%, 90%, 91%, 92%, 93%, 94%, 95%, 96%, 97%, 98% or 99% sequence identity to SEQ ID NO: 3.

Still alternatively, the hNEU1-coding sequence may include an intron in its nucleotide sequence. Depending on desired purpose, the intron may be derived from hNEU1 gene (i.e., a hNEU1 intron; SEQ ID NO: 15) or any genes other than hNEU1 gene (e.g., an beta-globin intron, SEQ ID NO: 14). According to certain exemplary embodiments, the hNEU1-coding sequence includes the hNEU1 intron of SEQ ID NO: 15 in its nucleotide sequence. In this case, the hNEU1-coding sequence comprises the nucleotide sequence of SEQ ID NO: 5. As known in the art, an intron refers to a nucleotide sequence in a nucleic acid that is excised through a process called RNA splicing and would not be expressed or operative in the mature RNA and the protein product translated therefrom. The intron may comprise one or more variations (e.g., nucleotide insertion, deletion and/or substitution) in its nucleotide sequence without affecting the activity or function of the protein product. Accordingly, the hNEU1-coding sequences comprising one or more variations in the intron region are also contemplated in the scope of the present disclosure. According to various embodiments of the present disclosure, the hNEU1-coding sequence may comprise a nucleotide sequence 85%, 86%, 87%, 88%, 89%, 90%, 91%, 92%, 93%, 94%, 95%, 96%, 97%, 98% or 99% identical to SEQ ID NO: 5.

According to certain embodiments, the hPPCA-coding sequence comprises the nucleotide sequence of SEQ ID NO: 6, and the thus-produced hPPCA protein comprises the amino acid sequence of SEQ ID NO: 7. As described above, a skilled artisan could substitute the nucleotides of the hPPCA-coding sequence with one or more degenerate codons or optimized codons in accordance with intended purpose, as long as the protein (i.e., the present hPPCA protein) encoded by the degenerate nucleotide sequence or optimized sequence maintains the desired activity or function. Accordingly, the nucleotide sequences comprising degenerate nucleotide(s) and/or optimized nucleotide(s) are intended to be included within the scope of the present disclosure, providing that the variations in the nucleotide sequence maintain at least 85% sequence identity to SEQ ID NO: 6, such as at least 85%, 86%, 87%, 88%, 89%, 90%, 91%, 92%, 93%, 94%, 95%, 96%, 97%, 98% or 99% sequence identity to SEQ ID NO: 6.

According to some preferred embodiments, the leader sequence (also known as “signal peptide” or “signal sequence”) of the hPPCA-coding sequence (SEQ ID NO: 6) is substituted with a leader sequence derived from human albumin (hAlb; SEQ ID NO: 8). In these embodiments, the hAlb-PPCA-coding sequence comprises the nucleotide sequence of SEQ ID NO: 9, and the thus-produced hAlb-hPPCA protein comprises the amino acid sequence of SEQ ID NO: 10. Based on the nucleotide degeneracy and codon optimization aforementioned, the hAlb-PPCA-coding sequence comprises variations (e.g., having a nucleotide sequence at least 85% identical to SEQ ID NO: 9) is also contemplated in the scope of the present disclosure. As could be appreciated, a skilled artisan may alternatively substitute the leader sequence of the hPPCA-coding sequence with a suitable leader sequence known to direct proteins to the secretory pathway, for example, the leader sequence of CD33, interleukin (IL)-2 or insulin.

Preferably, the expression cassette of the present disclosure further comprises, a bicistronic element disposed between the hNEU1-coding sequence and hPPCA-coding sequence as described above.

The bicistronic element may be an IRES, a 2A peptide, or any sequences or peptides known to express multiple proteins from one transcript. IRES is a sequence that recruits ribosomes and allows cap-independent translation. In practice, IRES serves as a linker linking two coding sequences in one bicistronic vector and allowing the translation of both proteins in cells. According to some exemplary embodiments, the bicistronic element is an IRES comprising the nucleotide sequence of SEQ ID NO: 16. According to alternative embodiments, the bicistronic element is an IRES comprising the nucleotide sequence of SEQ ID NO: 17. The 2A peptide also known as “2A self-cleaving peptide” is a class of peptide having 18 to 22 amino acid residues in length, which can induce ribosomal skipping during translation of a protein in cells. Examples of 2A peptide commonly used in the art include, but are not limited to, T2A (“EGRGSLLTCGDVEENPGP”; SEQ ID NO: 23), P2A (“ATNFSLLKQAGDVEENPGP”; SEQ ID NO: 24), E2A (“QCTNYALLKLAGDVESNPGP”; SEQ ID NO: 25) and F2A (“VKQTLNFDLLKLAGDVESNPGP”; SEQ ID NO: 26).

According to one preferred embodiment, the expression cassette of the present disclosure comprises, in the order from 5′ end to 3′ end, a human NEU1 P3 promoter (SEQ ID NO: 11), a hNEU1-coding sequence (SEQ ID NO: 1), IRES (SEQ ID NO: 16), and a hAlb-PPCA-coding sequence (SEQ ID NO: 9). In the embodiment, the expression cassette comprises the nucleotide sequence of SEQ ID NO: 22. According to various embodiments of the present disclosure, the expression cassette may comprise one or more degenerate nucleotides and/or optimized nucleotides in its nucleotide sequence without affecting the activity or function of the protein products (i.e., hNEU1 and hPPCA). Accordingly, the expression cassette comprising a nucleotide sequence at least 85% identical to SEQ ID NO: 22 also falls within the scope of the present disclosure.

According to certain embodiments, the expression cassette of the present disclosure may alternatively comprise, in the order from 5′ end to 3′ end, a human NEU1 P3 promoter (SEQ ID NO: 11), a hAlb-PPCA-coding sequence (SEQ ID NO: 9), IRES (SEQ ID NO: 16), and a hNEU1-coding sequence (SEQ ID NO: 1).

Optionally, the expression cassette of the present disclosure further comprises a beta-globin intron (SEQ ID NO: 14) disposed between the promoter and the hNEU1-coding sequence.

Preferably, the expression cassette of the present disclosure further comprises a polyA sequence disposed at the 3′ end of hPPCA-coding sequence as described above.

The polyA sequence (i.e., addition of a polyA tail to an RNA transcript) is important for the nuclear export, translation and stability of mRNA. According to some exemplary embodiments, the polyA sequence is derived from human growth hormone (hGH). In the embodiments, the polyA sequence comprises a nucleotide sequence at least 85% identical to SEQ ID NO: 18; preferably, at least 90% identical to SEQ ID NO: 18; more preferably at least 95% identical to SEQ ID NO: 18. In one specific embodiment, the polyA sequence comprises the nucleotide sequence of SEQ ID NO: 18. As could be appreciated, a skilled artisan may select a suitable polyA sequence in accordance with practical application, for example, the polyA sequence of bovine growth hormone (bGH), simian virus 40 (SV40), rabbit beta globin, or herpes simplex virus type 1 thymidine kinase (HSV-TK).

Optionally, the recombinant AAV vector of the present invention further comprises an enhancer close to the promoter. According to one embodiment, the recombinant AAV vector further comprises a CMV major immediate early enhancer (MIE) for to stimulate the expression of the hNEU1-coding sequence and hPPCA-coding sequence.

Optionally, the recombinant AAV vector of the present invention may further comprise a transcription initiation, termination, and/or efficient RNA processing signals, for example, splicing signal, regulatory element that enhance gene expression (e.g., woodchuck hepatitis virus post-transcriptional regulatory element (WPRE)), sequences that stabilize cytoplasmic mRNA, sequences that enhance translational efficiency (e.g., Kozak consensus sequence), sequences that enhance protein stability, and/or when desired, sequences that enhance the secretion of the encoded product.

The size of the recombinant AAV vector may be manipulated based on the size of the regulatory sequences, including the promoter, enhancer, intron, polyA, etc. According to some embodiments, the recombinant rAAV vector is about 4.4 kilobases (kb) to about 5.3 kb in size. According to one working example, the recombinant rAAV vector is about 4.7 kb in size.

### (ii) Recombinant AAV

The recombinant AAV vector described in Section (i) of the present disclosure is useful in producing a recombinant AAV via co-transfecting the recombinant AAV vector with a helper plasmid (an AAV plasmid carrying adenovirus-derived genes for AAV replication, including E2A, E4, and VA genes) and a Rep-Cap plasmid (a plasmid carrying Rep and Cap genes for viral replication and assemble) into a host cell (e.g., HEK293 cell). The thus-produced recombinant AAV comprises an AAV capsid and the recombinant AAV vector encapsulated in the AAV capsid. The methods of producing a recombinant AAV via co-transfection of three plasmids are known in the art; hence, the detailed description thereof is omitted herein for the sake of brevity.

The amino acid sequence of the AAV capsid is determined by the Cap sequence of the Rep-Cap plasmid. Preferably, the AAV capsid has a broad tropism for the brain and visceral organs. According to some preferred embodiments, the AAV capsid is derived from AAV9. In one working examples, the AAV capsid comprises the amino acid sequence of SEQ ID NO: 21.

(iii) Pharmaceutical Composition or Medicament Comprising the Recombinant AAV

According to working examples of the present disclosure, the recombinant AAV is useful in treating sialidosis via simultaneously expressing hNEU1 and hPPCA in the subject, without causing protein aggregation. Accordingly, another aspect of the present disclosure pertains to the use of the recombinant AAV in the preparation of a pharmaceutical composition or medicament for treating sialidosis. The pharmaceutical composition or medicament comprises the recombinant AAV as described in Section (ii) of the present disclosure, and a pharmaceutically acceptable carrier.

The pharmaceutically acceptable carrier may be any pharmaceutically acceptable material or vehicle, such as a liquid or solid filler, diluent, excipient, solvent or encapsulating material, that is useful in carrying or transporting the active agents (e.g., the present recombinant AAV) from one organ, or portion of the body, to another organ, or portion of the body. The carrier must be “acceptable” in the sense of being compatible with the other ingredients of the formulation, and is selected to minimize any degradation of the active agent and to minimize any adverse side effects in the subject. Depending on desired purposes, the pharmaceutical composition or medicament of the present disclosure may further comprise one or more pharmaceutically-acceptable additives, including surfactant, buffering agent, diluent, stabilizer, buffer, emulsifier, dispersing agent, suspending agent, preservative, and the like. The choice of the pharmaceutically acceptable carrier is basically determined by the way the pharmaceutical composition or medicament being administered. The pharmaceutical composition or medicament of the present invention may be administered to a subject via intra-cerebrospinal fluid or intravenous injection.

The pharmaceutical composition or medicament for administration by injection may be prepared in a sterile aqueous or non-aqueous solution, suspension, and emulsion. Examples of the non-aqueous solution include, but are not limited to, propylene glycol, polyethylene glycol, vegetable oils such as olive oil, and injectable organic esters such as ethyl oleate. Illustrative examples of the aqueous solution include water, emulsion, or suspension, such as saline and buffered media. Common parenteral vehicles include sodium chloride solution, Ringer's dextrose, dextrose, sodium chloride, lactated Ringer's, or fixed oils; whereas intravenous vehicles often include fluid and nutrient replenishes, electrolyte replenishes (such as those based on Ringer's dextrose), and the like. According to some working example, the carrier is phosphate buffered saline (PBS).

According to certain embodiments, the present pharmaceutical composition or medicament is produced in the form of an aqueous suspension suitable for administering to a sialidosis patient via intra-cerebrospinal fluid or intravenous injection. In some embodiments, the suspension contains at least 1×10¹³ genome copy (GC) of the recombinant AAV per milliliter (mL); preferably, about 1×10¹³ to 1×10¹⁵ GC of the recombinant AAV per mL. The genome copy of the recombinant AAV may be determined by any methods known in the art for viral quantification, such as quantitative PCR (qPCR), digital droplet PCR (ddPCR), or AAV vector genome (vg) titer assay.

### (iv) Uses of the Recombinant AAV, Pharmaceutical Composition or Medicament for Treating Sialidosis

Another aspect of the present disclosure pertains to a method of treating sialidosis in a subject. The method comprises administering to the subject an effective amount of the recombinant AAV, pharmaceutical composition or medicament in accordance with any aspect, embodiment of example of the present disclosure.

The recombinant AAV, pharmaceutical composition or medicament may be administered to the subject via any appropriate route, for example, intra-cerebrospinal fluid, intravenous, intraperitoneal, intraarterial or intramuscular injection.

According to some embodiments of the present disclosure, about 1×10¹³ to 1×10¹⁵ GC/kg brain weight of the recombinant AAV are administered to the subject, for example, 1×10¹³, 1.5×10¹³, 2×10¹³, 2.5×10¹³, 3×10¹³3.5×10¹³, 4×10¹³4.5×10¹³, 5×10¹³5.5×10¹³, 6×10¹³, 6.5×10¹³, 7×10¹³7.5×10¹³, 8×10¹³, 8.5×10¹³, 9×10¹³9.5×10¹³, 1×10¹⁴, 1.5×10¹⁴, 2×10¹⁴, 2.5×10¹⁴, 3×10¹⁴, 3.5×10¹⁴, 4×10¹⁴, 4.5×10¹⁴, 5×10¹⁴5.5×10¹⁴, 6×10¹⁴, 6.5×10¹⁴, 7×10¹⁴7.5×10¹⁴, 8×10¹⁴, 8.5×10¹⁴, 9×10¹⁴, 9.5×10¹⁴ or 1×10¹⁵ GC/kg of the recombinant AAV According to alternative embodiments, about 1×10¹³ to 1×10¹⁵ GC/kg body weight of the recombinant AAV are administered to the subject. As could be appreciated, the amount of the recombinant AAV administered to the subject may vary with clinical factors, such as age, gender, underlying diseases, treatment plan, conditioning regimen and infection. A skilled artisan or medical practitioner may adjust or optimize the administered amount of the recombinant AAV in accordance with desired purposes.

According to certain embodiments, the recombinant AAV, pharmaceutical composition or medicament is administered to the subject in a single dose. In one embodiment, the recombinant AAV, pharmaceutical composition or medicament is administered to cerebral spinal fluid (CSF) at a dosage about 1.0×10¹³ GC/kg brain weight to about 1.0×10¹⁵ GC/kg brain weight via lumbar puncture, cisternal puncture, or microcatheter that is navigated in the spinal canal from the lumbar region to the cisterna magna. In another embodiment, the CSF and intravenous administrations can be combined in a subject, in which the combined CSF and intravenous administrations can be performed concurrently or consecutively.

The viral particle administered to the subject varies with the yield of AAV in cell culture system. According to some embodiments, the average yield of AAV is about 50%, in which about 50% of the viral particle have a vector genome encapsulated in the capsid, i.e., about 50% full particles. Thus, for an exemplary dose of 1×10¹⁴ GC/kg, the total particle dose would be about 2×10¹⁴ GC/kg.

According to some embodiments of the present disclosure, the administration of the recombinant AAV exhibits a therapeutic effect on sialidosis in a subject (e.g., Neu1^{-/-} mice) via effectively expressing hNEU1 and hPPCA in the brain of the subject.

According to certain embodiments, the sialidosis is Type I sialidosis. According to certain embodiments, the sialidosis is Type II sialidosis.

The subject treatable by the present method is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

Neu1 knock-out (KO) mice (Neu1^{-/-})

Neu1^{-/-} mice were generated by CRISPR/Cas9 technology. In brief, wild-type C57BL/6J mouse zygotes were injected with Cas9 mRNA and two guide RNAs (sgRNAs; SEQ ID NOs: 27 and 28) targeting the first and third coding exons of Mus musculus Neu1 gene. Mice that developed from these embryos were sequenced to determine the deletion(s) and then bred with C57BL/6J mice to transmit the allele and confirm germline transmission. According to the sequencing results, Neu1^{-/-} mice contained a Neu1 deletion from middle exon 1 to middle exon 3, causing fusion of exons 1 and 3. All mice were maintained in a 12-hour light and dark cycle, provided fresh water and a standard mouse chow ad libitum.

The phenotypes of present Neu1^{-/-} mouse model were similar to those of the knockout mouse model of sialidosis as described by Natalie de Geest et al. (Systemic and neurologic abnormalities distinguish the lysosomal disorders sialidosis and galactosialidosis in mice, Hum Mol Genet., 2002; 11(12): 1455-64), in which the Neu1^{-/-} mice were smaller and had a shorter cranium than the wild-type mice (data not shown). The data of histological examination indicated the vacuolation in the kidney, bone marrow, and liver of 8-month-old Neu1^{-/-} mice (data not shown). Compared to normal control group, the Neu1^{-/-} mice exhibited decreased weight gain and motor dysfunction starting from 4 months of age, and usually died before they reaching 9 months of age (data not shown).

### Construction of the Recombinant AAV (AAV-P3-NP)

All sequences were derived from pAAV-MCS or produced via oligonucleotide synthesis technology. The gene therapy vector AAV-P3-hNEU1-IRES-hPPCA (AAV-P3-NP) was constructed by an AAV2 vector bearing a human NEU1 cDNA under the control of P3 NEU1 promoter, and a human PPCA cDNA with a human albumin leader peptide sequence linked to the human NEU1 cDNA via an IRES. The hNEU1-hPPCA expression cassette was flanked by AAV2 derived ITRs. The transgene also included the polyadenylation signal of human growth hormone (hGH). According to the sequencing results, the hNEU1-hPPCA expression cassette comprised the nucleotide sequence of SEQ ID NO: 22.

The expression of the constructed plasmid was determined by COS-1 cells. In brief, COS-1 cells were maintained in Dulbecco's Modified Eagle Medium (DMEM) with 10% fetal bovine serum (FBS). COS-1 cells in 6-cm dishes were subjected to transfection with the expression plasmids using LIPOFECTAMINE® 3000. 48 hours post-transfection, cells were fixed by paraformaldehyde for immunofluorescent staining, or lysed for enzyme activity measurements. For western blot analysis, cells were disrupted by sonication and centrifuged, and the supernatant and palette fractions were analyzed separately. The denatured proteins were separate by 10% polyacrylamide gel, blotted onto nylon paper, and incubated with primary and horseradish peroxidase (HRP)-labeled secondary antibodies.

For the purpose of producing the recombinant AAV, the bicistronic vector was co-transfected with a helper plasmid carrying adenovirus-derived genes for AAV replication (including E2A, E4, and VA genes) and a Rep-Cap plasmid (carrying Rep and Cap genes for viral replication and assemble) into HEK293 cells. The transfected cells were incubated at 37° C. for 72 hours. After centrifuging at 1,500 g for 5 minutes, the cell pellet was resuspended in 0.5 ml lysis buffer (10 mM Tris-HCl (pH8.5), 150 mM NaCl), followed by freeze/thaw treatment for 3 times through a dry ice/ethanol bath and a 37° C. water bath to obtain the crude lysate. The crude lysate was centrifuged at 3,000 g for 10 minutes. The supernatant was transferred to a new tube and proceed with purification to obtain the recombinant AAV.

### Animal Study

Gene therapy was conducted through two ways. For intracerebral ventricular (ICV) injection, 2 μl of recombinant AAV was injected to each lateral ventricle of neonatal mice (postnatal day 0, P0) at a dose of 2×10 GC/side. For intravenous (IV) injection, 30 μl of recombinant AAV was injected to the facial vein of neonatal mice (postnatal day 2, P2) at a dose of 5×10¹¹ GC/mouse. During viral injection, mice were placed on ice for anaesthetization. Viral vectors were injected with insulin needle to facial vein, or with HAMILTON® syringe to inject between lambda and bregma for ICV delivery. After injection, mice were placed under warm lamp until skin color return to normal.

For functional studies, rotarod test was performed on 3-cm-diameter spindles at five speeds (including 16, 20, 24, 28, and 32 rpm), in which mice were tested three times for each speed with a resting period of 5 minutes between each test. The data were averaged from the tested mice. A grip strength meter was used to measure the animals' limb grasping power. Mice were tested for three times for each test, with the best performance recorded.

### Immunohistochemistry (IHC) Staining

Mice were sacrificed and perfused with PBS containing 4% paraformaldehyde. Frozen 25 μm coronal sections through the bregma −1.5 to −2.0 mm, which contained the cerebral cortex, hippocampus and midbrain, were subjected to IHC staining after antigen retrieval and permeabilization. Dorsal root ganglions (DRG) were also isolated, pooled, and processed with a procedure similar to the brain tissues. The sections were incubated with primary antibodies diluted in blocking buffer at 4° C. overnight, followed by washing and incubating with fluorescence-labeled secondary antibodies at room temperature for 30 minutes. Nuclei were stained with 0.1 μg/ml DAPI (4′,6-diamidino-2-phenylindole) in PBS at room temperature for 5 minutes. The data was detected by confocal microscopy and analyzed by software.

For counting of LAMP1- or GFAP-positive cells, images of entire brain sections were acquired by an upright fluorescence and brightfield system at 20× magnification. These images were analyzed by software. In brief, after brightness/contrast adjustments and image alignment, regions of interest (ROIs) were defined manually, and signal area gating was applied to isolate specific areas with significant fluorescence signals.

### Statistics

Equal number of animals, n>5 if possible unless lost during follow up, were designed for each experiment. T-test was performed to compare the difference between two groups.

### Example 1 Characterization of hNEU1, hPPCA and Bicistronic Expression Cassette

For the purpose of examining the sialidase activity of hNEU1, three hNEU1-expression plasmids were constructed, including hNEU1 plasmid, hAlb-hNEU1 plasmid and mutant hNEU1 plasmid. The hNEU1 plasmid carried an authentic hNEU1 cDNA (SEQ ID NO: 1); the hAlb-hNEU1 plasmid carried an hNEU1 cDNA with an albumin leader sequence (hAlb-hNEU1); and the mutant hNEU1 plasmid carried an hNEU1 cDNA with mutations to decrease self-aggregation (mutant hNEU1; SEQ ID NO: 3). The plasmids were respectively transfected into COS-1 cells, and the sialidase activity of the transfected cells was determined. The data indicated that the authentic hNEU1 cDNA expressed modest sialidase activity (Table 1). Since over-expression of hNEU1 would result in self-aggregation, codon optimization to increase NEU1 protein expression may be not necessary.

TABLE 1
Expression of specified hNEU1 constructs

| | | | Sialidase |
|---|---|---|---|
| | Plasmid | DNA | (nmol/mg/h) |
| 1 | hNEU1 | 2 μg | 0.83 |
| 2 | hNEU1 | 4 μg | 1.30 |
| 3 | hAlb-hNEU1 | 2 μg | 2.04 |
| 4 | mutant hNEU1 | 2 μg | 0.45 |
| 5 | mock | — | 0.43 |

Also, four hPPCA-expression plasmids were constructed in the study, including hPPCA plasmid, hAlb-hPPCA plasmid, coPPCA plasmid and hAlb-coPPCA plasmid. The hPPCA plasmid carried an authentic hPPCA cDNA (SEQ ID NO: 6); the hAlb-hPPCA plasmid carried an hPPCA cDNA with an albumin leader sequence (hAlb-hPPCA; SEQ ID NO: 9); the coPPCA plasmid carried a codon optimized hPPCA (coPPCA); and the hAlb-coPPCA plasmid carried a codon optimized hPPCA with an albumin leader sequence (hAlb-coPPCA). The four hPPCA plasmids were respectively co-transfected with the hNEU1 plasmid or hAlb-hNEU1 plasmid into COS-1 cells, and the sialidase activity of the transfected cells was determined. As the results summarized in Table 2, hPPCA with an albumin leader peptide (i.e., hAlb-hPPCA) exhibited the highest sialidase activity as compared to other hPPCA constructs (Table 2)

TABLE 2
Sialidase activity of cells transfected with specified hNEU1 and hPPCA constructs

| Exp 1 | | | Sialidase | Exp 2 | | | Sialidase |
|---|---|---|---|---|---|---|---|
| disch | Plasmid 1 | Plasmid 2 | nmol/mg/hr | dish | Plasmid 1 | Plasmid 2 | nmol/mg/hr |
| 1 | hNEU1 | — | 1.94 | 1 | hNEU1 | — | 0.82 |
| 2 | hNEU1 | coPPCA | 2.31 | 2 | hNEU1 | — | 1.03 |
| 3 | hNEU1 | hAlb-hPPCA | 3.92 | 3 | hNEU1 | hAlb-hPPCA | 6.25 |
| 4 | hAlb-NEU1 | — | 1.90 | 4 | hNEU1 | hAlb-hPPCA | 5.73 |
| 5 | hAlb-NEU1 | coPPCA | 2.54 | 5 | hAlb-NEU1 | — | 2.01 |
| 6 | hAlb-NEU1 | hAlb-coPPCA | 4.43 | 6 | hAlb-NEU1 | hAlb-hPPCA | 5.75 |

Next, hNEU1 alone or hNEU1 plus hPPCA was expressed in COS-1 cells. The IHC results indicated that the overexpression of hNEU1 induced cell damage leading to morphological changes of cells; and co-expression of hPPCA protected COS-1 cells from the hNEU1-induced damage and improved cell morphology (data not shown). In COS-1 cells transfected with NEU1-expression plasmid, most of the expressed NEU1 protein was in the pellet fraction (data not shown). However, when the hPPCA-expression plasmid was added to the transfection, the expressed NEU1 protein was shifted to the soluble fraction (data not shown). The data demonstrated that the expression of hPPCA improved the solubility of hNEU1 protein.

After confirming the expression and activity of the present hNEU1 and hPPCA, a bicistronic vector comprising the hNEU1- and hAlb-hPPCA-coding sequences was constructed as described in “Materials and Methods”, in which the hNEU1- and hAlb-hPPCA-coding sequences were linked via an IRES element and were driven by a P3 promoter. The data of Table 3 demonstrated that the transfection of the bicistronic vector resulted in high sialidase activities in COS-1 cells. The modification of the construct by either an alternative IRES (IRES1; SEQ ID NO: 17) or by inserting an intron into the hNEU1-coding sequence did not further improve the sialidase activities (Table 4).

TABLE 3
Sialidase activity of cells transfected with specified constructs

| | | hAlb- | bicistronic | Sialidase |
|---|---|---|---|---|
| | hNEU1 | hPPCA | vector | (nmol/mg/hr) |
| 1 | 0.25 μg | | | 0.62 |
| 2 | 0.25 μg | | | 0.68 |
| 3 | 0.25 μg | 0.25 μg | | 9.50 |
| 4 | 0.25 μg | 0.25 μg | | 17.62 |
| 5 | | | 0.25 μg | 2.59 |
| 6 | | | 0.25 μg | 3.41 |
| 7 | | | 0.5 μg | 8.06 |
| 8 | | | 0.5 μg | 5.55 |
| 9 | Mock | | | 1.17 |

hNEU1: hNEU1 plasmid.
hAlb-hPPCA: hAlb-hPPCA plasmid.

TABLE 4
Sialidase activity of cells transfected with specified constructs

| | NEU1-IRES-PPCA | NEU-IRES1-PPCA | nmol/mg/hr |
|---|---|---|---|
| 1 | 0.5 ug | | 12.11 |
| 2 | 0.5 ug | | 10.04 |
| 3 | | 0.25 ug | 1.04 |
| 4 | | 0.25 ug | 1.06 |
| 5 | | 0.5 ug | 1.85 |
| 6 | | 0.5 ug | 2.03 |

NEU1-IRES-PPCA: the bicistronic vector comprising the hNEU1- and hAlb-hPPCA-coding sequences linked via the IRES of SEQ ID NO: 16.
NEU-IRES1-PPCA: the bicistronic vector comprising the hNEU1- and hAlb-hPPCA-coding sequences linked via the IRES1 of SEQ ID NO: 17.

The data of Table 5 further demonstrated that the P3 promoter was useful in driving the expression of hNEU1 and hPPCA in both COS-1 and N2A cells.

TABLE 5
Sialidase activity of cells transfected with specified constructs

| | DNA 0.5 μg each | Sialidase |
|---|---|---|
| | in COS-1 cells | (nmol/mg/hr) |
| 1 | CMV-NEU1, hAlb-hPPCA | 9.60 |
| 2 | CMV-NEU1, hAlb-hPPCA | 11.78 |
| 3 | P2-NEU1, hAlb-hPPCA | 4.31 |
| 4 | P2-NEU1, hAlb-hPPCA | 7.44 |
| 5 | P3-NEU1, hAlb-hPPCA | 5.09 |
| 6 | P3-NEU1, hAlb-hPPCA | 5.35 |
| 7 | Mock | 1.03 |
| 8 | Mock | 0.35 |

Next, the bicistronic vector was co-transfected with helper and Rep-Cap plasmids into HEK293 cells as described in “Materials and Methods”, so as to produce the recombinant AAV “AAV-P3-NP”. The data of Table 6 indicated that the viral genome containing the hNEU1-hPPCA expression cassette was successfully packaged in AAV9 capsid.

TABLE 6
Viral titer of specified recombinant AAV

| Name | Titer (GC/mL) |
|---|---|
| AAV-CMV-NP | 4.66 × 10¹² |
| AAV-P3-NP | 3.57 × 10¹³ |
| AAV-P3-GFP | 1.92 × 10¹³ |

AAV-P3-NP: the recombinant AAV produced by triple transfection of bicistronic vector, helper plasmid and Rep-Cap plasmid, in which the bicistronic vector comprised the hNEU1-hPPCA expression cassette driven by a P3 promoter.
AAV-CMV-NP: the recombinant AAV produced by triple transfection of bicistronic vector, helper plasmid and Rep-Cap plasmid, in which the bicistronic vector comprised the hNEU1-hPPCA expression cassette driven by a CMV promoter.
AAV-P3-GFP: the recombinant AAV produced by triple transfection of GFP vector, helper plasmid and Rep-Cap plasmid, in which the GFP vector comprised GFP gene driven by a P3 promoter.

### Example 2 In Vivo Study

### 2.1 Expression of the Present AAV-P3-NP in Neu1^{-/-} Mice

The recombinant AAV-P3-NP was administered to neonatal Neu1^{-/-} mice via intracerebral ventricular (ICV) or intravenous (IV) injection. Three months post-injection, the brain tissues were isolated from the mice. Thin slices of the brain tissues were subjected to IHC analysis. According to the IHC results, PPCA signal was present but NEU1 signal was absent in untreated Neu1^{-/-} mice, and large amount of LAMP1 was accumulated in IbaI-positive microglia cells (data not shown). By contrast, wide-spread expression of hNEU1 was observed in ICV-injected Neu1^{-/-} mice, including cerebral cortex, hippocampus, putamen, brain stem, and cerebellum (data not shown). It was further observed that the perikaryon of microglia was positive for hNEU1 staining, and no hNEU1 signal was detected in astrocytes (data not shown). hPPCA staining was also observed, though weaker than hNEU1 staining (data not shown). Double staining for hNEU1 and hPPCA indicated that these two proteins could be co-expressed in the cells of Neu1^{-/-} mice (data not shown). Further, the results of IHC analysis demonstrated that most of the hNEU1-positive cells were NeuN-positive, suggesting that hNEU1 was expressed mainly in neurons after the ICV administration of the present AAV-P3-NP (data not shown).

These data suggested that the specificity of AAV-P3-NP was similar to the natural biodistribution of NEU1 protein in the brain.

### 2.2 Effect of the Present AAV-P3-NP on Neu1^{-/-} Mice

Whether the present AAV-P3-NP may serve as a therapeutic agent for treating sialidosis was evaluated in this example. As described in Materials and Methods, Neu1^{-/-} mice, a mouse model of sialidosis, were administered with the present AAV-P3-NP via ICV or IV injection. PBS served as a negative control in the study. The effect of the present AAV-P3-NP on sialidosis was determined by biochemical and histological analyses and functional tests.

### 2.2.1 Biochemical and Histological Analyses

Half brain and liver tissues of one-month-old wild-type, Neu1^{-/-}, or ICV-injected Neu1^{-/-} mice were subjected to enzyme activity analysis. There was a severe reduction of sialidase activity in the brain, and a partial deficiency of sialidase activity in the liver of Neu1^{-/-} mice, and ICV injection of the present AAV-P3-NP normalized sialidase activity in the brain (data not shown). There was an elevation of β-galactosidase activity in the brain and liver of Neu1^{-/-} mice, and ICV injection of the present AAV-P3-NP decreased β-galactosidase activity in both the brain and liver of the mice (data not shown).

Long-term correction of brain pathologies by gene therapy were observed in 8-month-old IV- or ICV-injected Neu1^{-/-} mice. Brain regions including cortex, hippocampus, and midbrain were analyzed by immunofluorescence for LAMP1, NEU1, and GFAP. The data indicated that the number of astrocytes (with GFAP staining) increased in all regions of the brain of Neu1^{-/-} mice (a pathology known as “astrogliosis”, a common phenomenon observed in lysosomal storage diseases with brain involvement), while there was no significant change in the number of microglia in the brain of Neu1^{-/-} mice (data not shown). According to the results, the morphology of these astrocytes was not altered, and no LAMP1 (a membranous marker for the lysosome) was accumulated in the astrocytes. Further, IHC analysis with anti-LAMP1 antibody revealed prominent engorged lysosomes in microglia all over the brain, in which these microglia were round in shape, a sign for activation (data not shown). The administration of AAV-P3-NP via ICV- or IV-injection inhibited the proliferation of astrocyte in Neu1^{-/-} mice (data not shown). Compared to the activated microglia with swollen and large cell body as observed in Neu1^{-/-} mice, the microglia of AAV-P3-NP-treated Neu1^{-/-} mice exhibited normal morphology, and there was no LAMP1 staining in the brain of Neu1^{-/-} mice treated with AAV-P3-NP (data not shown). In the treated Neu1^{-/-} mice, NEU1 signal increased, while LAMP1 and GFAP signals decreased. Whole brain section cell counting revealed that both ICV- and IV-injection of the present AAV-P3-NP lowered the number of LAMP1-positive cells, while only ICV injection of the present AAV-P3-NP decreased the number of GFAP-positive cells (data not shown). The data suggested that the AAV-P3-NP treatment improved the pathological changes in the brain of Neu1^{-/-} mice.

Dorsal root ganglion (DRG) specimens from untreated 8-month-old Neu1^{-/-} mice were also analyzed. The data indicated that more than half of the neurons in DRG exhibited prominent LAMP1 signal (data not shown). However, in IV-injected Neu1^{-/-} mice, the proportion of LAMP1-positive neurons decreased (data not shown). The DRG specimens from wild-type and Neu1^{-/-} mice were further stained with antibodies specific to peripherin (nociceptor marker) or neurofilament (mechanoreceptor marker). According to the results, the shape and cell number of peripherin- or neurofilament-positive neurons were similar between the wild-type and Neu1^{-/-} mice, regardless of the intense LAMP1 staining in the Neu1^{-/-} mice (data not shown).

### 2.2.2 Functional Tests

Compared to the wild-type control mice, Neu1^{-/-} mice exhibited less weight gain and deteriorating performance in rotarod test at both 24 and 32 rpm, starting from 4 months of age (data not shown). Further, Neu1^{-/-} mice had deficits in grasping power since young age (data not shown). The administration of AAV-P3-NP via intracerebral ventricular or intravenous injection improved the body weight, the performance in rotarod test, and the grasping power of Neu1^{-/-} mice. According to the results of rotarod test, compared with untreated Neu1^{-/-} mice, the ICV-injected Neu1^{-/-} mice showed benefit of treatment at 28 rpm (p<0.01) at 7 months, and at 24 rpm (p<0.001), 28 rpm (p<0.0001) and 32 rpm (p<0.0001) at 8 months; the facial vein-injected Neu1^{-/-} mice showed benefit of treatment at 24 rpm (p<0.05) at 6 months, at 28 rpm (p<0.001) and 32 rpm (p<0.05) at 7 months, and at 28 rpm (p<0.01) and 32 rpm (p<0.001) at 8 months. In the fore-limb grip strength test, compared with untreated Neu1^{-/-} mice, the facial vein-injected Neu1^{-/-} mice showed benefit of treatment at 2 months (p<0.01), 3 months (p<0.0001), 4 months (p<0.001), 5 months (p<0.05), and 7 months (p<0.05). In the four-limb grip strength test, the facial vein-injected Neu1^{-/-} mice showed benefit of treatment at 3 months (p<0.05). Of note, the recombinant AAV did not replicate with cell division; accordingly, the administration of the present AAV-P3-NP during neonatal periods would be largely lost after cell proliferation in somatic tissues including the muscles. Therefore, the improvements in motor function were assumed to be attributed to the therapeutic effects in the nervous system.

The body weight was further analyzed according to the sex of mice. Compared with the male mice, the female Neu1^{-/-} mice tended to have a lower body weight and were less responsive to treatment (data not shown). In the rotarod test (at 32 rpm), the female mice tended to have a poorer performance (data not shown). Unfortunately, statistics could not be applied because of small sample sizes after dividing the mice into two groups.

These results suggested that the administration of AAV-P3-NP is useful in improving the pathological changes, body weight and exercise performance of Neu1^{-/-} mice (an animal model of sialidosis known in the art that develops a systemic and neurodegenerative condition of sialidosis as observed in human patients). Accordingly, the present AAV-P3-NP may provide a potential means to treat sialidosis.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A recombinant viral vector comprising an AAV 5′ inverted terminal repeat (ITR) sequence, an AAV 3′ ITR sequence, and an expression cassette, wherein the expression cassette is flanked by the AAV 5′ ITR and 3′ ITR sequences, and comprises,
(a) a promoter;
(b) a first coding sequence operably linked to the promoter encoding a human neuraminidase 1 (NEU1); and
(c) a second coding sequence operably linked to the promoter encoding a human protective protein/cathepsin A (PPCA).

2. The recombinant viral vector of claim 1, wherein the AAV 5′ ITR and 3′ ITR sequences are derived from AAV serotype 2 (AAV2).

3. The recombinant viral vector of claim 2, wherein the AAV 5′ ITR and 3′ ITR sequences respectively comprise the nucleotide sequences of SEQ ID NOs: 19 and 20.

4. The recombinant viral vector of claim 1, wherein the promoter is a human NEU1 promoter or cytomegalovirus (CMV) promoter.

5. The recombinant viral vector of claim 4, wherein the promoter comprises the nucleotide sequence of SEQ ID NO: 11.

6. The recombinant viral vector of claim 1, wherein the human NEU1 and human PPCA respectively comprise the amino acid sequences of SEQ ID NOs: 2 and 10.

7. The recombinant viral vector of claim 6, wherein the first and second coding sequences respectively comprise the nucleotide sequences of SEQ ID NOs: 1 and 9.

8. The recombinant viral vector of claim 1, wherein the expression cassette further comprises an internal ribosomal entry site (TRES) disposed between the first and second coding sequences.

9. The recombinant viral vector of claim 1, wherein the expression cassette further comprises a third coding sequence disposed between the first and second coding sequences, wherein the third coding sequence is operably linked to the promoter and encodes a 2A peptide.

10. The recombinant viral vector of claim 1, wherein the expression cassette further comprises a polyadenylation (polyA) sequence disposed at the 3′ end of the second coding sequence.

11. The recombinant viral vector of claim 10, wherein the polyA sequence is derived from human growth hormone (hGH).

12. The recombinant viral vector of claim 1, wherein the expression cassette comprises the nucleotide sequences of SEQ ID NO: 22.

13. A recombinant adeno-associated virus (AAV) comprising an AAV capsid and the recombinant viral vector of claim 1 packaged therein.

14. The recombinant AAV of claim 13, wherein the AAV capsid is derived from AAV serotype 9 (AAV9).

15. The recombinant AAV of claim 14, wherein the AAV capsid comprises the amino acid sequence of SEQ ID NO: 21.

16. (canceled)

17. A method of treating sialidosis in a subject, comprising administering to the subject an effective amount of the recombinant AAV of claim 13, so as to alleviate or ameliorate the symptoms of the sialidosis.

18. The method of claim 17, wherein about 1×10¹³ to 1×10¹⁵ genome copy (GC)/kg brain weight of the recombinant AAV is administered to the subject.

19. The method of claim 17, wherein about 1×10¹³ to 1×10¹⁵ genome copy (GC)/kg body weight of the recombinant AAV is administered to the subject.

20. The method of claim 17, wherein the recombinant AAV is administered to the subject via intra-cerebrospinal fluid or intravenous injection.

21. The method of claim 17, wherein the subject is a human
